(19) United States
(12) Patent Application Publication
(10) Pub. No.: US 2017/0220782 A1
(43) Pub. Date: Aug. 3, 2017

(54) MOBILE INTERFACE PLATFORM SYSTEMS AND METHODS

(71) Applicant: Ali ALSANOUSI, Boston, MA (US)

(72) Inventor: Ali ALSANOUSI, Boston, MA (US)

(21) Appl. No.: 15/490,592

(22) Filed: Apr. 18, 2017

Related U.S. Application Data
(63) Continuation-in-part of application No. 14/480,315, filed on Sep. 8, 2014.

(51) Int. Cl. G06F 21/12; A61B 5/00; G06F 9/455; H04W 12/06; G06F 21/57

(52) U.S. Cl. CPC G06F 21/126; H04W 12/06; G06F 21/57; G06F 9/45558; A61B 5/0022; G06F 2221/0715; G06F 19/322

(57) ABSTRACT

Mobile Health Interface (mHi) Platform systems and methods of evaluating mobile applications include establishing evaluation criteria for mobile applications within a given industry; receiving mobile applications with associated Application Programming Interfaces (APIs) for the given industry; classifying, via the APIs, each of the mobile applications into discreet packages; certifying and accepting the discreet packages for each of the mobile applications based upon the evaluation criteria, via processing circuitry of a single interoperable platform with data integration capability and associated virtual machine; authenticating a user access to the certified discreet packages for a set trial period of time; receiving trial sequence data indicating the user's preference via scoring for each of the certified discreet packages for the set period of time; ranking the certified discreet packages based upon the received trial sequence data; and receiving a selected bundle of certified and ranked discreet packages.

### CROSS REFERENCE TO RELATED APPLICATIONS

This application is a continuation-in-part of U.S. Ser. No. 14/480,315, filed Sep. 8, 2014, which is incorporated in its entirety by reference herein.

### BACKGROUND

Field of the Disclosure

Systems and methods of forming a mobile interface platform are described. In particular, a mobile interface platform of evaluated and validated mobile applications is described herein. Still further, a mobile health interface platform of evaluated and validated mobile health applications is described.

Description of the Related Art

The “background” description provided herein is for the purpose of generally presenting the context of the disclosure. Work of the presently named inventor, to the extent it is described in this background section, as well as aspects of the description which may not otherwise qualify as prior art at the time of filing, are neither expressly nor impliedly admitted as prior art against the present disclosure.

Mobile applications are currently being built independently, with little or no data sharing. In contrast, the use of mobile applications is becoming multi-dimensional and evolving. A full or complete picture of mobile applications for a particular industry will require integrating, processing, and visualizing data from multiple applications and data sources.

### SUMMARY

Embodiments include a method of comparing mobile applications. The method includes establishing evaluation criteria for mobile applications within a given industry; receiving a plurality of mobile applications with associated Application Programming Interfaces (APIs) for the given industry; classifying, via the APIs, each of the mobile applications into discreet packages; certifying and accepting one or more of the discreet packages for each of the mobile applications based upon the evaluation criteria, via processing circuitry of a single interoperable platform with data integration capability and associated virtual machine; authenticating a user access to the certified discreet packages of the plurality of mobile applications for a set trial period of time; receiving trial sequence data indicating the user's preference for each of the certified discreet packages for the set period of time; ranking, via the processing circuitry, the certified discreet packages based upon the received trial sequence data; and receiving a selected bundle of ranked and certified discreet packages.

Embodiments also include a mobile application network. The network includes an interoperable platform and associated virtual machine; an operating system connected to the interoperable platform; a server connected to the operating system; and processing circuitry configured to establish evaluation criteria for mobile applications within a given industry, receive a plurality of mobile applications with associated APIs within the given industry, classify, via the APIs, each of the mobile applications into discreet packages, certify and accept one or more of the discreet packages for each of the mobile applications based upon the evaluation criteria, via the interoperable platform with data integration capability and associated virtual machine, authenticate a user access to the certified discreet packages of the plurality of mobile applications for a set trial period of time, receive trial sequence data indicating the user's preference for each of the certified discreet packages for the set period of time, rank the certified discreet packages based upon the received trial sequence data integrated with data from the evaluation criteria, and receive a selected bundle of ranked and certified discreet packages.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 illustrates a networking system with multiple application types, according to an embodiment;

FIG. 2 illustrates networking systems directed to an industry type of application, according to an embodiment;

FIG. 3A illustrates an architectural framework, according to an embodiment;

FIG. 3B illustrates a mobile health interface (mHi) platform architectural framework, according to an embodiment;

FIG. 4 is a block diagram illustrating an exemplary electronic device according to an embodiment;

FIG. 5 is a block diagram of a computing system, according to an embodiment;

FIG. 6 is a schematic diagram of a data processing system according to an embodiment;

FIG. 7 is a block diagram of a CPU according to an embodiment;

FIG. 8 is an illustration of a cloud computing system according to an embodiment;

FIG. 9 is a flowchart of a method of comparing mobile applications, according to an embodiment;

FIG. 10 is a pictorial illustration of a method of comparing mobile applications according to an embodiment; and

FIG. 11 is an illustration of a user interface according to an embodiment.

Referring now to the drawings, wherein like reference numerals designate identical or corresponding parts throughout the several views.

### DETAILED DESCRIPTION

Mobile health, as used herein refers, in part to the incorporation of mobile telecommunication, multimedia technology, and mobile communication devices, as well as any other device used to communicate health-related matters via wireless communication for delivery of medical health services and clinical trials services. Examples of mobile health devices include, but are not limited to glucometers, pulse oximeters, pedometers, sphygmomanometers, biofeedback devices, urine analyzers, and pulmonary function test devices. These devices may be used in or out of a medical setting and may communicate wirelessly with each other.

Mobile health is an area of growth driven in part by the increasing use of mobile health products, such as mobile computing platforms, mobile health applications that run on mobile computing platforms, and peripherals. Physicians and other healthcare professionals utilize mobile computing platforms loaded with mobile health applications to improve patient care. Mobile health applications can be used to help healthcare providers more accurately estimate and calculate healthcare parameters, illustrate and explain health conditions to patients, access and edit electronic health records, and utilize peripherals, such as probes, meters, or other mobile health hardware for diagnostic purposes.

Patients can also use mobile health products to help manage particular medical conditions or their overall wellbeing. Some mobile health products are advisory in nature, such as dealing with first aid or weight management. Other mobile health products provide guidance with a medication, or monitoring health items such as glucose levels or heart rates. Some mobile health products also communicate data to physicians in real-time.

The proliferation of healthcare data, especially generated by mobile health technology requires new ways of integrating them together to be useful for patients, physicians, researchers, and public health officials. There is a need for a mobile health platform and architecture that will bridge between health and technology to enable collaboration and provide a holistic view of a patient's health.

A large number and variety of software-based medical applications have been developed by academic and commercial entities for use in a variety of areas in the medical field, including patient diagnostics, results reporting, treatment planning, post-procedure follow up, and clinical operations. Medical applications can provide immediate or convenient access to laboratory or imaging tests, provide evidence-based clinical decision-making tools, or address operational efficiencies in healthcare by reducing paperwork or providing logistical support.

In a related system and method, Happtique describes retrieving patient information, along with physician and/or insurance information to send the patient a recommended mobile health product. An online mobile health product store can be used to fill the mobile health product order. An associated database can collect patient information for a related clinical condition. Mobile health products can be suggested by a decision engine (see U.S. Patent Publication No. 20130066650). Happtique also describes various technical standards for testing mobile application (see “Technical testing of healthcare mobile apps, a request for proposals (RFP)”, which is incorporated in its entirety by reference herein). However, Happtique does not describe separating a mobile application into discreet independent packages, which can be individually offered to a user.

Information used by a clinician or a specialist for a patient evaluation or consultation is often based on data from one or more prior consultations, or from one or more previous diagnostic tests. However, data collected from one clinician or medical system may be incompatible or incomplete for use by another clinician or medical system. Healthcare informatics attempts to deal with this by merging information science, computer science, and health care to optimize, among other things, the acquisition, storage, retrieval, display, or use of information in healthcare or biomedicine.

A mobile health application should easily link together with other similar applications. An objective of embodiments described herein includes aggregating multiple types of data from multiple types of applications together, such that the patient's relevant data is integrated into a single application. Conversely, basic health data could auto-populate multiple applications. For example, most applications require certain basic information, such as gender, height, weight, and date of birth. Instead of repeating the same information for each new mobile health application, the information could be automatically populated into each subsequent application with the authorization and permission of the associated user, via electronic user consent, for example even though the applications may be very different from one another. A common data format could provide interoperability within the different applications. An example of implementing the sharing of common data could request the user to take the data from one application and populate the information requests into another application. This would eliminate the need for repeated entry of common data.

Embodiments described herein include a platform in which mobile applications are interconnected and can be exchanged. The platform is designed on open standards to accept multiple types of mobile applications that are built on different mobile platforms, such as Apple iOS, Google Android, Samsung Bada, etc.

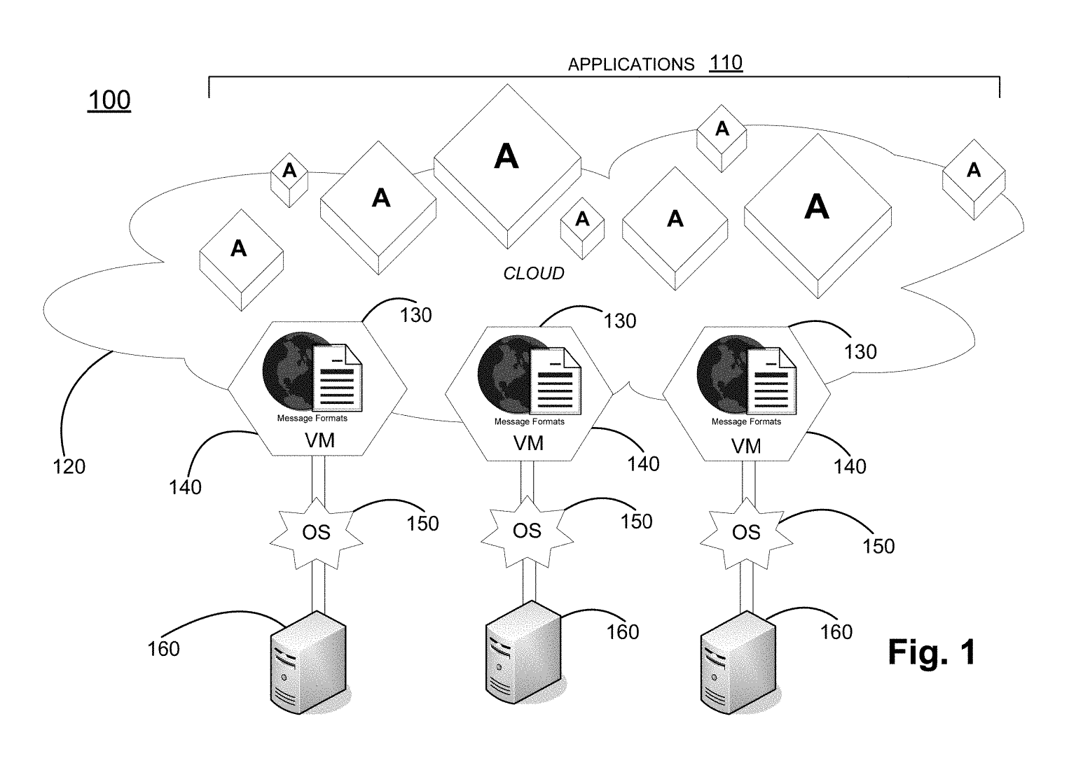

Embodiments described herein for a mobile health application platform enable data integration from multiple and varied applications. FIG. 1 illustrates a networking system 100 in which different types of mobile applications are used. Several applications 110 exist from all types of industries. Applications 110 from the health, auto, music, entertainment, food, exercise, education, and travel may be present, in addition to medical and health applications, to name just a few. The applications 110 are connected, via a cloud or network 120 to multiple platforms 130. Since the platforms 130 are not interoperable, several platforms 130 may be necessary to handle the various types of applications 110 present. Each platform 130 contains its own version of Java Virtual Machine (JVM) 140. However, other virtual machine languages which can run on multiple hardware/operating system platforms are contemplated by embodiments described herein. The individual JVMs 140 do not share data and therefore, are not integrated together. Multiple Operating Systems (OSs) 150 are present to operate with their associated platforms 130 and JVMs 140. Individual servers 160 are also present for each OS 150.

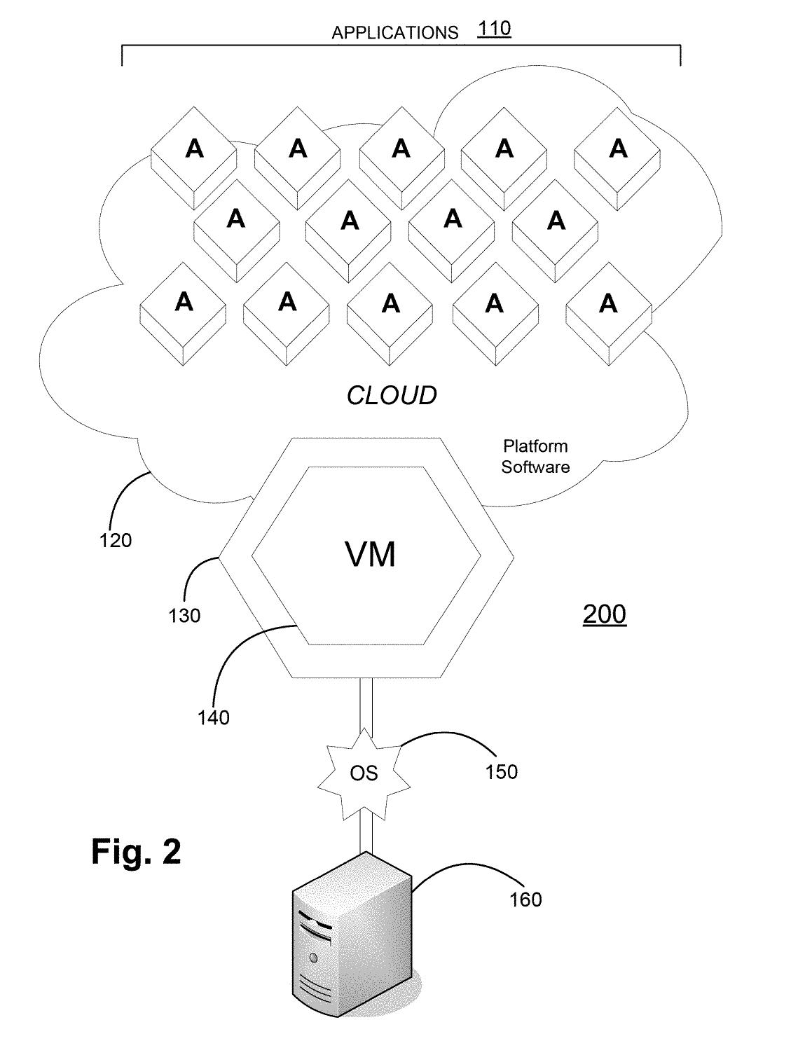

FIG. 2 illustrates an interoperable service-oriented platform as part of a contrasting and simpler system 200 to aggregate and integrate mobile applications. As illustrated in FIG. 2, applications 110 are aggregated into a specific industry, such as the health and medical industry. The specific industry applications 110 are connected, via the cloud or network 120 to a single interoperable and service-oriented platform 130. A single JVM 140 (or other virtual machine language) operates within the single platform 130. Since there is just one platform 130, there is a need for just one OS 150 and one server system 160. Applications 110 share a basic API with the platform 130 to integrate the applications 110 and their services into the platform 130. A common data format provides data integration within the applications 110. In an example, a user would allow data from one application 110 to populate information requests from another application 110, thereby eliminating repeated entry of common data.

Any other general industry could be represented by a system similar to system 200. There could be other similar systems for any type of industry, such as the auto, music, entertainment, food, exercise, education, and travel industries, to name just a few. Any group of industry-specific applications 110 has the advantage of being integrated into a single platform 130 governed by a single JVM 140. Each group of industry-specific applications 110 also requires just a single OS 150 and a single server system 160. As a result, a common data format provides integration for multiple applications 110 within an associated industry.

In an embodiment, system 200 includes circuitry configured to receive a plurality of mobile applications and their associated APIs for a particular industry. Each mobile application is separated into discreet packages via each associated API. A discreet package is a discreet feature of a mobile application which can function as a separate entity, apart from other discreet packages within the same mobile application. For example, a first medical-related mobile application pertaining to heart disease might be separated into a data gathering discreet package, a disease information discreet package, a drug information discreet package, and an exercise discreet package. A second medical-related mobile application pertaining to hormone replacement therapy might be separated into a nutrition discreet package, a natural medicine discreet package, and a drug risk discreet package.

Separating a mobile application into discreet packages allows a user to select only the discreet packages of interest, instead of requiring purchase of an entire mobile application. A user could select one or more discreet packages from a plurality of mobile applications. This provides a user with only the features of interest and at a reduced price, since the user is not required to purchase an entire mobile application.

In another embodiment, system 200 includes circuitry configured to receive relevant microdata from a plurality of mobile applications and integrate the microdata onto a single mobile application. The circuitry is further configured to receive an indication as to whether one or more discreet packages of an application have been approved by a governing body as evaluation criteria, and output the approved one or more discreet packages of the application to an interface connected to a server from which the discreet packages can be transmitted when the discreet packages have been approved by the governing body.

The circuitry is further configured to receive from the server, information related to a user's state or condition, such as a medical condition. Based upon the state or condition, the circuitry is configured to output a recommendation for one or more mobile applications and/or one or more relevant service applications.

In another embodiment, system 200 also includes circuitry configured to receive from a server information related to the user's state or condition and output the received information which corresponds to data fields in a mobile application. The circuitry is also configured to determine at a host device when the mobile application is received by the host device and determine data fields that are currently unpopulated. The circuitry is further configured to obtain data corresponding to the unpopulated fields from the server of a stored electronic record.

Most industries strive to have some type of utility measurement—a tool by which a consumer can help determine whether a particular application has utility for the user. One reliable utility test is usage, wherein an application with a large number of users would tend to indicate that the application provides utility. Most utility measurements involve a five-star ranking system. This provides some measurement of utility, but it is limited in scope.

Embodiments described herein provide a much more direct participation approach for measuring utility and value of a mobile application. One embodiment allows a user to “test drive” two applications within a family of related applications. The first application is active for a limited time, after which time it becomes inactive. The second application becomes active for the same amount of time. The user is allowed to purchase which of the two applications he/she prefers. If neither application satisfies the user, the user can continue to test other applications until a satisfactory one is found. Over time with several users “test driving” several applications, a ranking is developed amongst the different applications. In another embodiment, a user is allowed to “test drive” different discreet packages of one or more applications.

In another embodiment, system 200 includes circuitry configured to receive an authentication for a user to access a plurality of mobile applications containing de-identified data sets for a predetermined trial period. Following the trial period, the circuitry is configured to receive pairwise trial sequence data indicating a user's preference for each of the plurality of mobile applications or discreet packages of one or more application with respect to every other application within the plurality of mobile applications. The circuitry is further configured to generate a ranking for each of the plurality of mobile applications based on the pairwise trial sequence data.

Several evaluation criteria could be developed for a particular industry for the platform to accept as a safe and accurate application. General evaluation criteria could be applied across multiple industries. The evaluation criteria is preset or pre-designed, or previously agreed upon by experts of the particular industry to determine a ranking of new applications with respect to existing applications on the platform. As a result, applications will constantly be shifting upwards or downwards, based upon the latest rankings. The evaluation can be conducted by experts within the particular industry and/or an industry-related standards body that have been approved by the platform.

Evaluation criteria can be judged from a technical review and a content review. For example, the mission should be defined to determine whether the application addresses the defined goals and how well the defined goals are addressed. The application should be designed for ease of use to present the information in a way that is easy for target audiences to use. The application should be innovative, interesting, and unique in meeting the application requirements. The application should make an impact to achieve an industry objective in a creative way. The application should work correctly to achieve participant objectives. The application should have a likelihood of continued use for the intended population.

Table 1 illustrates an exemplary evaluation criteria rating of a general mobile application. In Table 1, ratings range from one to five, in which a rating of one designates a strong dislike or disagreement with the associated criteria. A rating of five designates a strong liking or agreement with the associated criteria. Table 1 is given for illustrative purposes only. Several other criteria and associated rating formats are contemplated by embodiments described herein.

TABLE 1
Evaluation Criteria for Rating a Mobile Application

| | Dislike or | | | | Like or |
|---|---|---|---|---|---|
| | Disagree | | Neutral | | Agree |
| Criteria | 1 | 2 | 3 | 4 | 5 |

| | | | | |
|---|---|---|---|---|
| Is the mobile app easy to use? | | | X | |
| Are the instructions clear and | | X | | |
| simple? | | | | |
| Did you find what you needed? | X | | | |
| Were the results presented | | | X | |
| nicely? | | | | |
| Is the mobile app creative? | | | | X |
| Is the mobile app interesting? | | | X | |
| Does the mobile app meet one or | | X | | |
| more industry objectives? | | | | |
| Would you use this mobile app | | X | | |
| again? | | | | |
| Would you recommend this | | | X | |
| mobile app to others? | | | | |
| What is your overall impression? | | | X | |

For a medical mobile application, evaluation criteria should be judged from multiple perspectives, such as a doctor perspective and a patient perspective. A medical mobile application can be reviewed for credibility, usability, patient safety, accuracy, performance, interactivity, and design. Evaluation criteria for a doctor can include, but are not limited to clinical assistance in making a diagnosis, remote monitoring of a patient, productivity in making the doctor's job more efficient, and provide references. Evaluation criteria for a patient can include, but are not limited to personalization, healthy living, and meaningful reminders and alerts.

Table 2 illustrates an exemplary evaluation criteria rating of a mobile health application. Evaluation criteria have been separated into physician evaluation criteria and patient evaluation criteria. The review sections have been separated into content review and technical review.

TABLE 2
Evaluation Criteria of Mobile Health Applications

| | CONTENT REVIEW | | | | TECHNICAL REVIEW | | |
|---|---|---|---|---|---|---|---|
| | Credibility | Usability | Patient Safety | Accuracy | Performance | Interactivity | Design |
| Physician Evaluation Criteria | | | | | | | |
| Clinical/assistance in diagnosis | X | ✓ | ? | X | X | X | X |
| Remote monitoring | X | X | ? | X | X | ✓ | X |
| Productivity | X | ✓ | ✓ | X | ✓ | ✓ | X |
| References | ✓ | ✓ | ✓ | X | ✓ | X | X |
| Overall impression | [symbol] | [symbol] | [symbol] | [symbol] | [symbol] | [symbol] | [symbol] |
| Patient Evaluation Criteria | | | | | | | |
| Personalization | X | X | X | X | X | X | X |
| Healthy Living | ✓ | ✓ | X | X | ✓ | X | ✓ |
| Meaningful reminders/alerts | X | X | X | X | X | ✓ | ✓ |
| Overall Impression | [symbol] | [symbol] | [symbol] | [symbol] | [symbol] | [symbol] | [symbol] |

Legend
✓ = Available
X = Not available
? = No data [symbol]
Established [symbol]
Starting [symbol]
Lacking

Criteria could also be developed for a particular subset or sub-cluster of applications. For the medical and health industry as an example, a sub-cluster could be applications related to blood pressure and/or heart rate monitoring. Criteria for the user to evaluate might include clear instructions, user-friendly input, organized format, length of time to complete, reliability of connected products (e.g. blood pressure kit), and real-time results pushed to a third party (e.g. doctor's office). Each criterion could be ranked from most important to least important, along with each criterion's rating from a user. An analogy that depicts this concept is an athletic ranking of several teams competing in a sport over a regular season. Criteria could include the number of wins versus losses, errors, assists, the number of tie games, a strength of each schedule, and playing time, all of which could be ranked. Each team could have their points for each criterion matched against the respective criterion ranking. This would result in that team's ranking amongst all other teams.

Several methods or models are available to evaluate a product or service. Multiple-Criteria Decision-Making (MCDM) or Multiple-Criteria Decision Analysis (MCDA) is a discipline of operations research that explicitly considers multiple criteria in decision-making environments. One MCDM method is called Potentially All Pairwise Rankings of All Possible Alternatives (PAPRIKA). PAPRIKA is used to calculate point values or weights on the criteria or attributes for decision problems involving ranking, prioritizing, or choosing between alternatives. Point values represent the relative importance of the criteria, and are used to rank alternatives. The PAPRIKA method specifically applies to additive multi-attribute value models with performance categories. Additive multi-attribute value models have multiple criteria with two or more performance categories within each criterion, which are combined additively. Each category is worth a certain number of points that is intended to reflect both the relative importance of the criterion and its degree of achievement. For each alternative being considered, the point values are summed across the criteria to get a total score, by which the alternatives are prioritized or ranked relative to each other.

A second MCDM method is called Multi-Attribute Global Inference of Quality (MAGIQ). MAGIQ is based on a hierarchical decomposition of comparison attributes and rating assignment using rank order centroids. The MAGIQ technique is used to assign a single, overall measure of quality to each member of a set of systems where each system has an arbitrary number of comparison attributes. The MAGIQ process begins with an evaluator determining which system attributes or criteria are to be used as the basis for system comparison. These criteria are ranked by importance to the particular problem domain, and the ranks are converted to ratings using rank order centroids. Each system under analysis is rated against each comparison criterion and the ratings are transformed into rank order centroids. The final overall quality metric for each system is the weighted sum of each criterion rating.

A third MCDM method is called Measuring Attractiveness by a Categorical Based Evaluation Technique (MACBETH). MACBETH is an interactive approach that requires only qualitative judgments about differences to help a decision maker or a decision-advising group quantify the relative attractiveness of options. It employs an initial, interactive, questioning procedure that compares two elements at a time, requesting a qualitative preference judgment. As judgments are entered, a numerical scale is generated that is entirely consistent with all the decision maker's judgments, through which process weights are generated for the criteria.

Several other methods and models are available by which data can be ranked, including methods and models to compare data pairwise, in a list-wise correlation, or through multiple alignment. The three MCDM methods described above are exemplary, and embodiments described herein are not limited to any of these three methods. The three MCDM methods described above do not have any implicit or explicit order of preference.

An objective of the present disclosure is to adequately and completely rank a mobile application that will give users confidence in the application, so they can make an informed decision. Embodiments described herein provide a method of receiving an authentication to access a plurality of mobile applications containing de-identified data sets for a predetermined trial period. Following the trial period, trial sequence data is received, which indicates a user's preference for each of the plurality of applications with respect to every other application. A ranking for each of the mobile applications is generated, which is based on the trial sequence data. Acceptance of a mobile application into a particular industry platform will be based upon the calculated ranking according to applicable criteria. The described ranking can also be used to rank discreet packages of an application.

Table 3 illustrates an exemplary ranking of multiple discreet packages A through E of a mobile application App 1, such as a mobile health application. Ratings ranged from a score of one for a poor rating to five for an excellent rating. Trial sequence data is received from users for topics including easy to use, clear instructions, presentation of results, and interesting. Evaluation criteria data is also received from professionals and/or organizations within a particular industry. Topics included remote monitoring, productivity, references, and managing assistance. Each discreet package received a combined rating score to determine the ranking of each discreet package for a mobile application.

TABLE 3
Ranking of a Mobile Application

Discreet Packages

| “App 1” | “A” | “B” | “C” | “D” | “E” |
|---|---|---|---|---|---|
| User Ratings | | | | | |
| Easy to use | 2 | 5 | 4 | 4 | 5 |
| Clear instructions | 2 | 4 | 3 | 3 | 4 |
| Presentation | 4 | 4 | 3 | 5 | 3 |
| Interesting | 2 | 3 | 5 | 4 | 2 |
| Professional Ratings | | | | | |
| Remote monitoring | 3 | 1 | 2 | 4 | 3 |
| Productivity | 4 | 5 | 3 | 3 | 2 |
| References | 4 | 5 | 5 | 3 | 4 |
| Managing assistance | 2 | 4 | 3 | 3 | 3 |
| Total rating score | 23 | 31 | 28 | 29 | 26 |
| Ranking of “App 1” | 5 | 1 | 3 | 2 | 4 |

Table 3 illustrates that Discreet Package “B” ranked first, followed by Discreet Package “D” ranking second, Discreet Package “C” ranking third, Discreet Package “E” ranking fourth, and Discreet Package “B” ranking last. Table 3 illustrates a simplified ranking process for ease of illustration. However, a large abundance of mobile applications and their respective discreet packages are within embodiments described herein. In addition, each ranked discreet package is active in real time. Therefore, the ranking of any discreet package can increase or decrease at any time with additional ratings received and from new certified applications entering the platform.

Another embodiment of the present disclosure includes evaluation criteria from a third party validation. An example of a third party validation is a Food and Drug Administration (FDA) approval rating. Several government and organized groups have well-established and well-known standards by which a product or service is approved or rated. A mobile application would display a greater confidence and/or utility to a prospective user when it has been approved using the systems and methods described herein, including a third-party validation or endorsement.

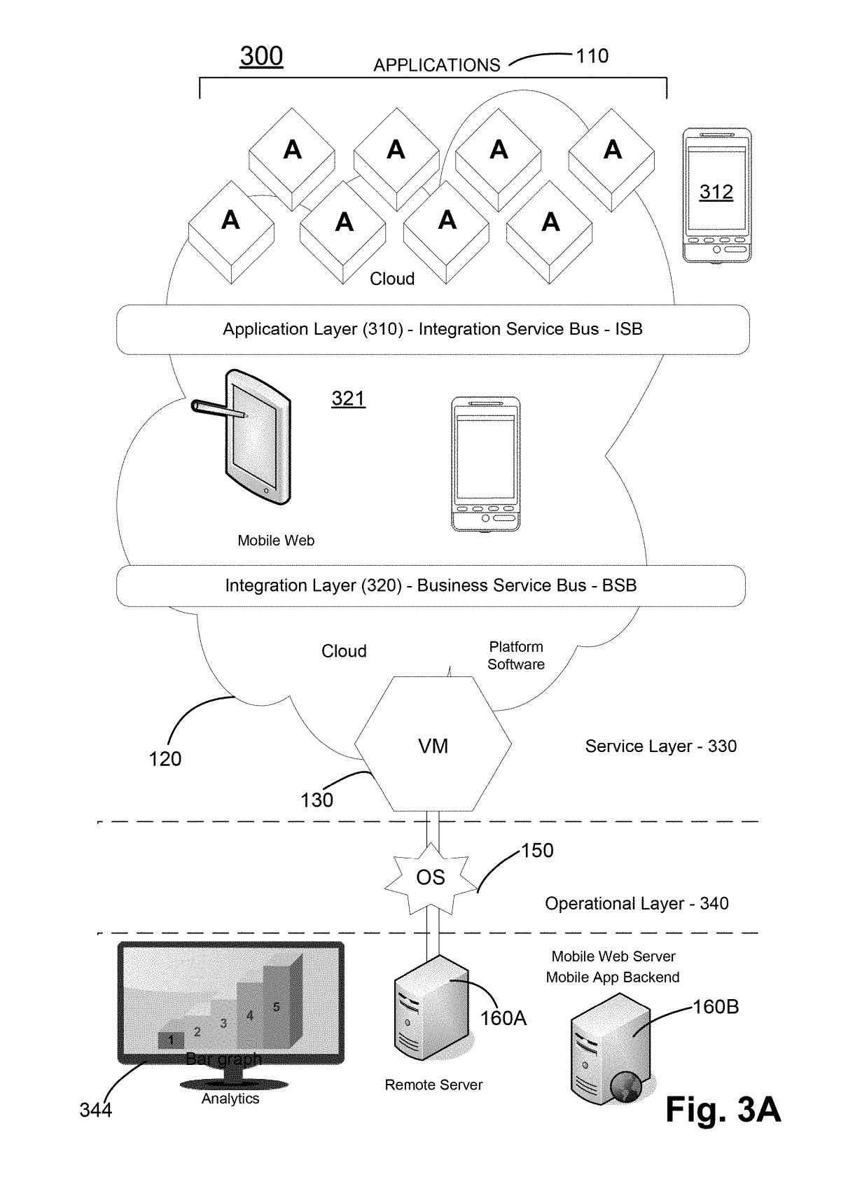

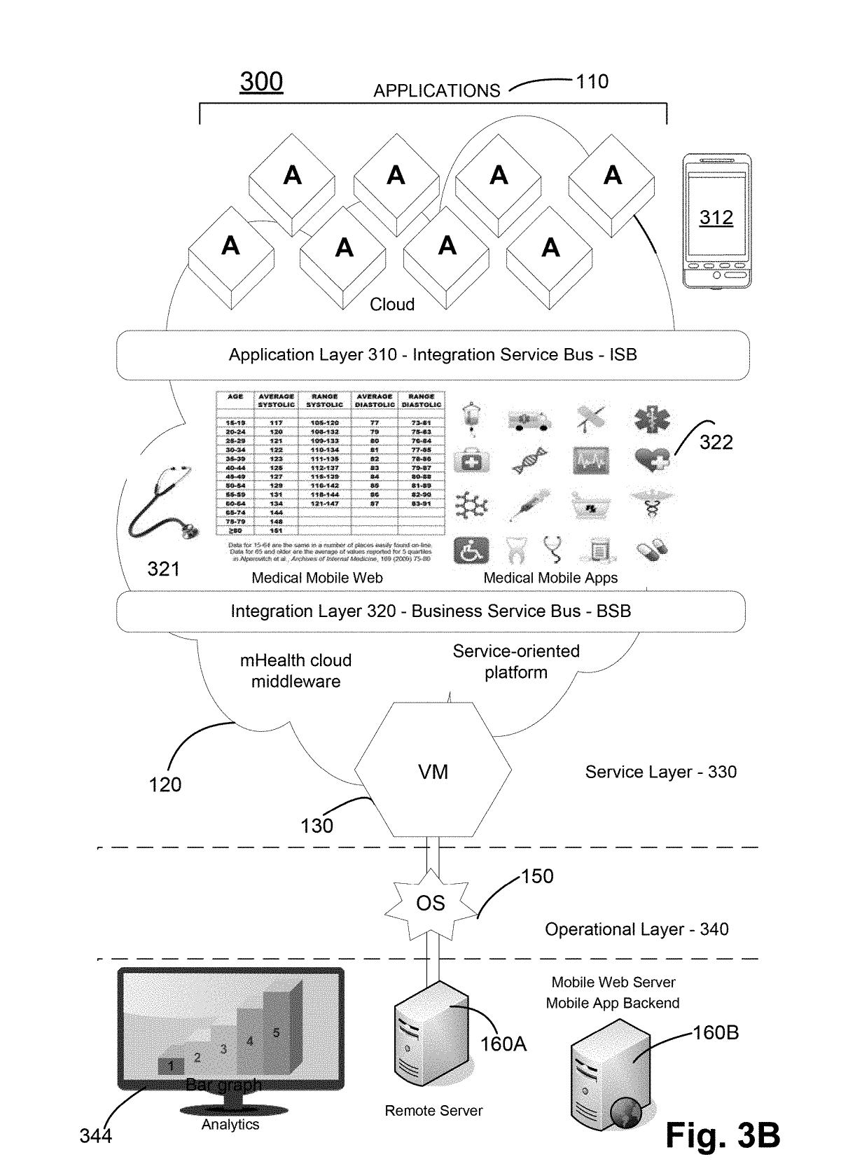

FIG. 3A illustrates an architectural framework 300 by which embodiments described herein could be implemented. An application layer 310 is illustrated, in which multiple mobile applications 110 for a specific industry, such as the health and medical industry are available for downloading to a mobile device 312. The architectural framework 300 is applicable for any other type of industry, such as the auto, music, entertainment, food, exercise, education, and travel industries, to name just a few.

An integration layer 320 is illustrated in which mobile tools and products are available to a user, such as mobile web 321.

A service layer 330 is illustrated, in which the applications 110 are interconnected, via the cloud 120 to the interoperable platform 130 and associated JVM. The mobile applications 110 share some features of their individual APIs with the platform 130 in order to integrate discreet packages of the applications 110 and their services with the platform 130.

An operational layer 340 is illustrated, by which the applications 110 are made available to a user via operating system 150 and a remote server 160A. A mobile web server 160B and an analytics center 344 of a mobile application backend infrastructure are illustrated. The mobile application backend infrastructure includes a universal database tool containing an integrated development environment for database query, administration, and development.

FIG. 3B illustrates an architectural framework 300 for a mobile Health interface (mHi) platform. Medical-oriented mobile web 321 and medical-oriented mobile applications 322 are illustrated. A service-oriented platform and mobile Health (mHealth) cloud middleware are illustrated. mHealth cloud middleware can include, but is not limited to HIPAA regulations, business logic, analytics, and regulations for sales, storage of data, and security. Architectural framework 300 illustrates a relationship between mobile devices, mobile cloud middleware, cloud services, healthcare systems such as an Electronic Medical Record (EMR) or an Electronic Health Record (EHR), and personal user systems such as patient portals. It provides an open platform to allow innovation through integration.

Many mHealth applications require user-specific information to provide the intended utility. Data might include the gender, height, weight, and date of birth of the user. The same data needs to be entered each time a new application is purchased. The mHealth application provides support for a common data format to allow data integration within multiple applications. When prompted, a user allows data from one application to populate information requested from another application, thereby eliminating repeated entry of common data.

Over time, an mHealth marketplace will expand and be more inclusive. Certain medical conditions tend to catalyze a cluster of applications around a particular condition. For example, a diabetic cluster can include a glucose monitoring application, a diet application, a social networking application, and a medical supplies ordering application. To facilitate clustering, the mHealth marketplace can provide cluster-specific suggestions that match profiles of similar users.

In an embodiment, the mHealth marketplace can provide personalized Electronic Health Records (pEHR) storage. The pEHR gathers new medical data collected with each new application download. If a user downloads a new application that requires information from the user that had not been requested previously, such as blood type, the pEHR expands its data collection for that user by one new field, i.e. blood type. The pEHR makes it available to subsequent applications when requested, if approved by the user. The bi-directional nature prevents the need to create an EHR that anticipates all information requests before the need arises via electronic user consent, for example.

Mobile medical applications need to be credible for physicians to prescribe the applications and be confident in recommending them to their patients for use. The FDA has started to regulate the accreditation of a mobile medical application using a risk-based approach to ensure safety. Unfortunately, not all applications originate in the United States or apply for FDA approval, and the wait time can be long. In contrast, embodiments described herein include an mHi platform in which every application has been approved using evaluation criteria for the applicable industry.

The mHealth marketplace can provide a real-time accumulation of a person's health history. For example, a user can input medical data, such as sleeping habits, a mood disturbance, blood pressure and pulse, or other signs and/or symptoms into an associated mobile medical application. In turn, the inputted data can be available to an attending physician and/or provider to better understand the user's medical status and provide an informed and up-to-date diagnosis. The bi-directional nature of data provides identification and automatic dissemination for both the patient and the care provider.

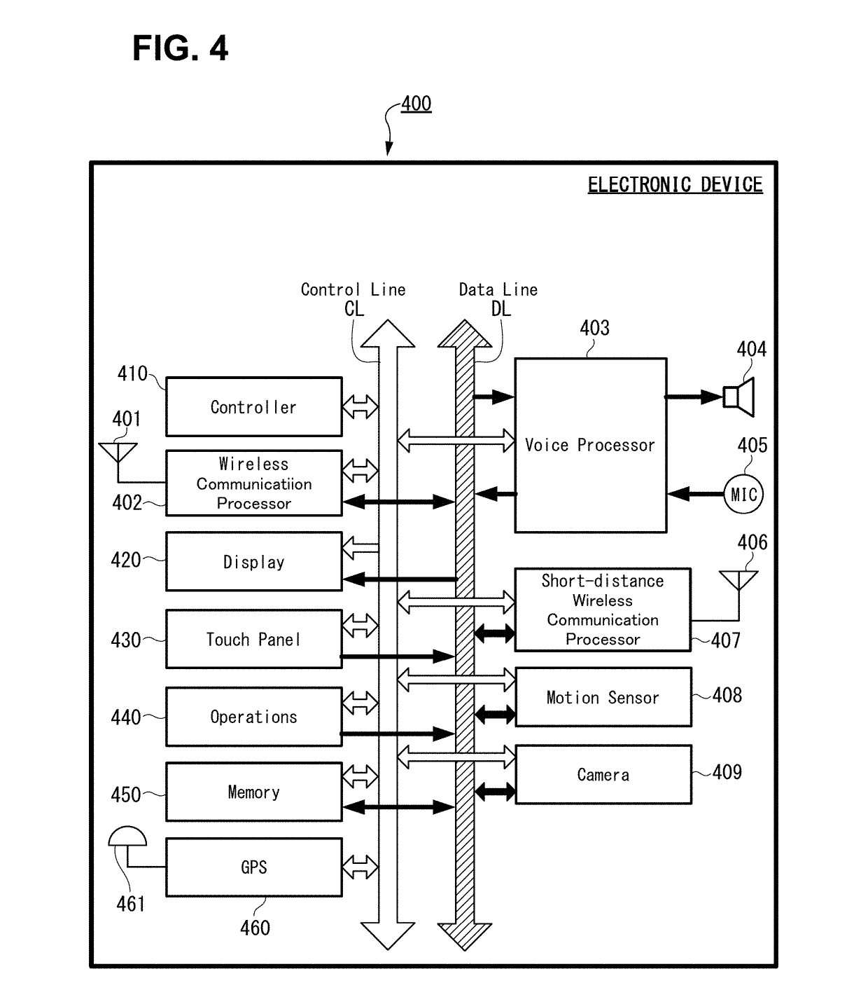

FIG. 4 is a block diagram illustrating an exemplary electronic device used in accordance with embodiments of the present disclosure. In some embodiments, electronic device 400 can be a smartphone, a laptop, a tablet, a server, an e-reader, a camera, a navigation device, etc. Electronic device 400 could be used as one or more of the mobile devices 312 in FIGS. 3A and 3B. The exemplary electronic device 400 of FIG. 4 includes a controller 410 and a wireless communication processor 402 connected to an antenna 401. A speaker 404 and a microphone 405 are connected to a voice processor 403.

The controller 410 can include one or more Central Processing Units (CPUs), and can control each element in the electronic device 400 to perform functions related to communication control, audio signal processing, control for the audio signal processing, still and moving image processing and control, and other kinds of signal processing. The controller 410 can perform these functions by executing instructions stored in a memory 450. Alternatively or in addition to the local storage of the memory 450, the functions can be executed using instructions stored on an external device accessed on a network or on a non-transitory computer readable medium.

The memory 450 includes but is not limited to Read Only Memory (ROM), Random Access Memory (RAM), or a memory array including a combination of volatile and non-volatile memory units. The memory 450 can be utilized as working memory by the controller 410 while executing the processes and algorithms of the present disclosure. Additionally, the memory 450 can be used for long-term storage, e.g., of image data and information related thereto.

The electronic device 400 includes a control line CL and data line DL as internal communication bus lines. Control data to/from the controller 410 can be transmitted through the control line CL. The data line DL can be used for transmission of voice data, display data, etc.

The antenna 401 transmits/receives electromagnetic wave signals between base stations for performing radio-based communication, such as the various forms of cellular telephone communication. The wireless communication processor 402 controls the communication performed between the electronic device 400 and other external devices via the antenna 401. For example, the wireless communication processor 402 can control communication between base stations for cellular phone communication.

The speaker 404 emits an audio signal corresponding to audio data supplied from the voice processor 403. The microphone 405 detects surrounding audio and converts the detected audio into an audio signal. The audio signal can then be output to the voice processor 403 for further processing. The voice processor 403 demodulates and/or decodes the audio data read from the memory 450 or audio data received by the wireless communication processor 402 and/or a short-distance wireless communication processor 407. Additionally, the voice processor 403 can decode audio signals obtained by the microphone 405.

The exemplary electronic device 400 can also include a display 420, a touch panel 430, an operations key 440, and a short-distance communication processor 407 connected to an antenna 406. The display 420 can be a Liquid Crystal Display (LCD), an organic electroluminescence display panel, or another display screen technology. In addition to displaying still and moving image data, the display 420 can display operational inputs, such as numbers or icons which can be used for control of the electronic device 400. The display 420 can additionally display a GUI for a user to control aspects of the electronic device 400 and/or other devices. Further, the display 420 can display characters and images received by the electronic device 400 and/or stored in the memory 450 or accessed from an external device on a network. For example, the electronic device 400 can access a network such as the Internet and display text and/or images transmitted from a Web server.

The touch panel 430 can include a physical touch panel display screen and a touch panel driver. The touch panel 430 can include one or more touch sensors for detecting an input operation on an operation surface of the touch panel display screen. The touch panel 430 also detects a touch shape and a touch area. Used herein, the phrase “touch operation” refers to an input operation performed by touching an operation surface of the touch panel display with an instruction object, such as a finger, thumb, or stylus-type instrument. In the case where a stylus or the like is used in a touch operation, the stylus can include a conductive material at least at the tip of the stylus such that the sensors included in the touch panel 430 can detect when the stylus approaches/contacts the operation surface of the touch panel display (similar to the case in which a finger is used for the touch operation).

According to aspects of the present disclosure, the touch panel 430 can be disposed adjacent to the display 420 (e.g., laminated) or can be formed integrally with the display 420. For simplicity, the present disclosure assumes the touch panel 430 is formed integrally with the display 420 and therefore, examples discussed herein can describe touch operations being performed on the surface of the display 420 rather than the touch panel 430. However, the skilled artisan will appreciate that this is not limiting.

For simplicity, the present disclosure assumes the touch panel 430 is a capacitance-type touch panel technology. However, it should be appreciated that aspects of the present disclosure can easily be applied to other touch panel types (e.g., resistance-type touch panels) with alternate structures. According to aspects of the present disclosure, the touch panel 430 can include transparent electrode touch sensors arranged in the X-Y direction on the surface of transparent sensor glass.

The touch panel driver can be included in the touch panel 430 for control processing related to the touch panel 430, such as scanning control. For example, the touch panel driver can scan each sensor in an electrostatic capacitance transparent electrode pattern in the X-direction and Y-direction and detect the electrostatic capacitance value of each sensor to determine when a touch operation is performed. The touch panel driver can output a coordinate and corresponding electrostatic capacitance value for each sensor. The touch panel driver can also output a sensor identifier that can be mapped to a coordinate on the touch panel display screen. Additionally, the touch panel driver and touch panel sensors can detect when an instruction object, such as a finger is within a predetermined distance from an operation surface of the touch panel display screen. That is, the instruction object does not necessarily need to directly contact the operation surface of the touch panel display screen for touch sensors to detect the instruction object and perform processing described herein. Signals can be transmitted by the touch panel driver, e.g. in response to a detection of a touch operation, in response to a query from another element based on timed data exchange, etc.

The touch panel 430 and the display 420 can be surrounded by a protective casing, which can also enclose the other elements included in the electronic device 400. According to aspects of the disclosure, a position of the user's fingers on the protective casing (but not directly on the surface of the display 420) can be detected by the touch panel 430 sensors. Accordingly, the controller 410 can perform display control processing described herein based on the detected position of the user's fingers gripping the casing. For example, an element in an interface can be moved to a new location within the interface (e.g., closer to one or more of the fingers) based on the detected finger position.

Further, according to aspects of the disclosure, the controller 410 can be configured to detect which hand is holding the electronic device 400, based on the detected finger position. For example, the touch panel 430 sensors can detect a plurality of fingers on the left side of the electronic device 400 (e.g., on an edge of the display 420 or on the protective casing), and detect a single finger on the right side of the electronic device 400. In this exemplary scenario, the controller 410 can determine that the user is holding the electronic device 400 with his/her right hand because the detected grip pattern corresponds to an expected pattern when the electronic device 400 is held only with the right hand.

The operation key 440 can include one or more buttons or similar external control elements, which can generate an operation signal based on a detected input by the user. In addition to outputs from the touch panel 430, these operation signals can be supplied to the controller 410 for performing related processing and control. According to aspects of the disclosure, the processing and/or functions associated with external buttons and the like can be performed by the controller 410 in response to an input operation on the touch panel 430 display screen rather than the external button, key, etc. In this way, external buttons on the electronic device 400 can be eliminated in lieu of performing inputs via touch operations, thereby improving water-tightness.

The antenna 406 can transmit/receive electromagnetic wave signals to/from other external apparatuses, and the short-distance wireless communication processor 407 can control the wireless communication performed between the other external apparatuses. Bluetooth, IEEE 802.11, and Near-Field Communication (NFC) are non-limiting examples of wireless communication protocols that can be used for inter-device communication via the short-distance wireless communication processor 407.

The electronic device 400 can include a motion sensor 408. The motion sensor 408 can detect features of motion (i.e., one or more movements) of the electronic device 400. For example, the motion sensor 408 can include an accelerometer to detect acceleration, a gyroscope to detect angular velocity, a geomagnetic sensor to detect direction, a geo-location sensor to detect location, etc., or a combination thereof to detect motion of the electronic device 400. According to aspects of the disclosure, the motion sensor 408 can generate a detection signal that includes data representing the detected motion. For example, the motion sensor 408 can determine a number of distinct movements in a motion (e.g., from start of the series of movements to the stop, within a predetermined time interval, etc.), a number of physical shocks on the electronic device 400 (e.g., a jarring, hitting, etc., of the electronic device 400), a speed and/or acceleration of the motion (instantaneous and/or temporal), or other motion features. The detected motion features can be included in the generated detection signal. The detection signal can be transmitted, e.g., to the controller 410, whereby further processing can be performed based on data included in the detection signal. The motion sensor 408 can work in conjunction with a Global Positioning System (GPS) 460. The GPS 460 detects the present position of the electronic device 400. The information of the present position detected by the GPS 460 is transmitted to the controller 410. An antenna 461 is connected to the GPS 460 for receiving and transmitting signals to and from a GPS satellite.

Electronic device 400 can include a camera 409, which includes a lens and shutter for capturing photographs of the surroundings around the electronic device 400. In an embodiment, the camera 409 captures surroundings of an opposite side of the electronic device 400 from the user. The images of the captured photographs can be displayed on the display panel 420. A memory saves the captured photographs. The memory can reside within the camera 409 or it can be part of the memory 450. The camera 409 can be a separate feature attached to the electronic device 400 or it can be a built-in camera feature.

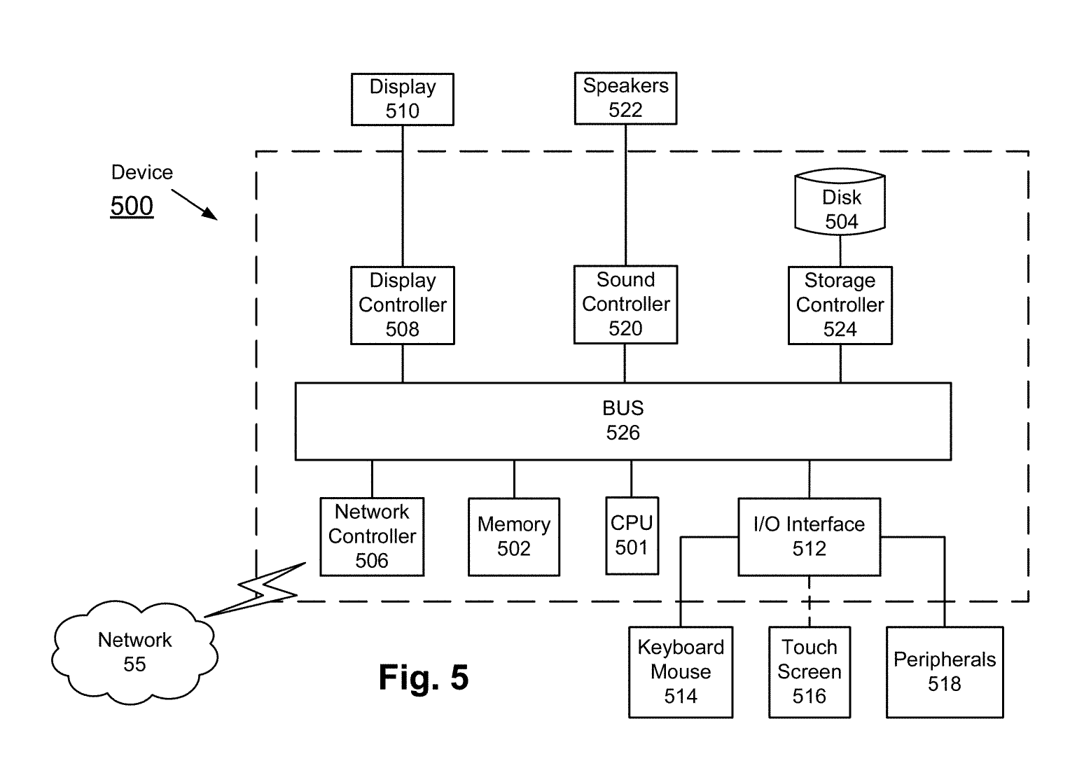

A hardware description of a computing device 500 used in accordance with exemplary embodiments is described with reference to FIG. 5. One or more features described above with reference to electronic device 400 of FIG. 4 can be included in computing device 500 described below. Computing device 500 could be used as one or more of the devices illustrated in servers 160,160A, and 160B.

In FIG. 5, the computing device includes a CPU 501 which performs the processes described above. The process data and instructions may be stored in memory 502. These processes and instructions may also be stored on a storage medium disk 504 such as a Hard Disk Drive (HDD) or portable storage medium or may be stored remotely. Further, the claimed embodiments are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computing device communicates, such as a server or computer.

Further, the claimed embodiments may be provided as a utility application, background daemon, or component of an operating system, or combination thereof, executing in conjunction with CPU 501 and an operating system such as Microsoft Windows 7, UNIX, Solaris, LINUX, Apple MAC-OS and other systems known to those skilled in the art.

CPU 501 may be a Xenon or Core processor from Intel of America or an Opteron processor from AMD of America, or may be other processor types that would be recognized by one of ordinary skill in the art. Alternatively, the CPU 501 may be implemented on an FPGA, ASIC, PLD or using discrete logic circuits, as one of ordinary skill in the art would recognize. Further, CPU 501 may be implemented as multiple processors cooperatively working in parallel to perform the instructions of the inventive processes described above.

The computing device 500 in FIG. 5 also includes a network controller 506, such as an Intel Ethernet PRO network interface card from Intel Corporation of America, for interfacing with network 55. As can be appreciated, the network 55 can be a public network, such as the Internet, or a private network such as an LAN or WAN network, or any combination thereof and can also include PSTN or ISDN sub-networks. The network 55 can also be wired, such as an Ethernet network, or can be wireless such as a cellular network including EDGE, 3G and 4G wireless cellular systems. The wireless network can also be WiFi, Bluetooth, or any other wireless form of communication that is known.

The computing device 500 further includes a display controller 508, such as a NVIDIA GeForce GTX or Quadro graphics adaptor from NVIDIA Corporation of America for interfacing with display 510, such as a Hewlett Packard HPL2445w LCD monitor. A general purpose I/O interface 512 interfaces with a keyboard and/or mouse 514 as well as a touch screen panel 516 on or separate from display 510. General purpose I/O interface 512 also connects to a variety of peripherals 518 including printers and scanners, such as an OfficeJet or DeskJet from Hewlett Packard.

A sound controller 520 is also provided in the computing device, such as Sound Blaster X-Fi Titanium from Creative, to interface with speakers/microphone 522 thereby providing sounds and/or music.

The general purpose storage controller 524 connects the storage medium disk 504 with communication bus 526, which may be an ISA, EISA, VESA, PCI, or similar, for interconnecting all of the components of the computing device 500. A description of the general features and functionality of the display 510, keyboard and/or mouse 514, as well as the display controller 508, storage controller 524, network controller 506, sound controller 520, and general purpose I/O interface 512 is omitted herein for brevity as these features are known.

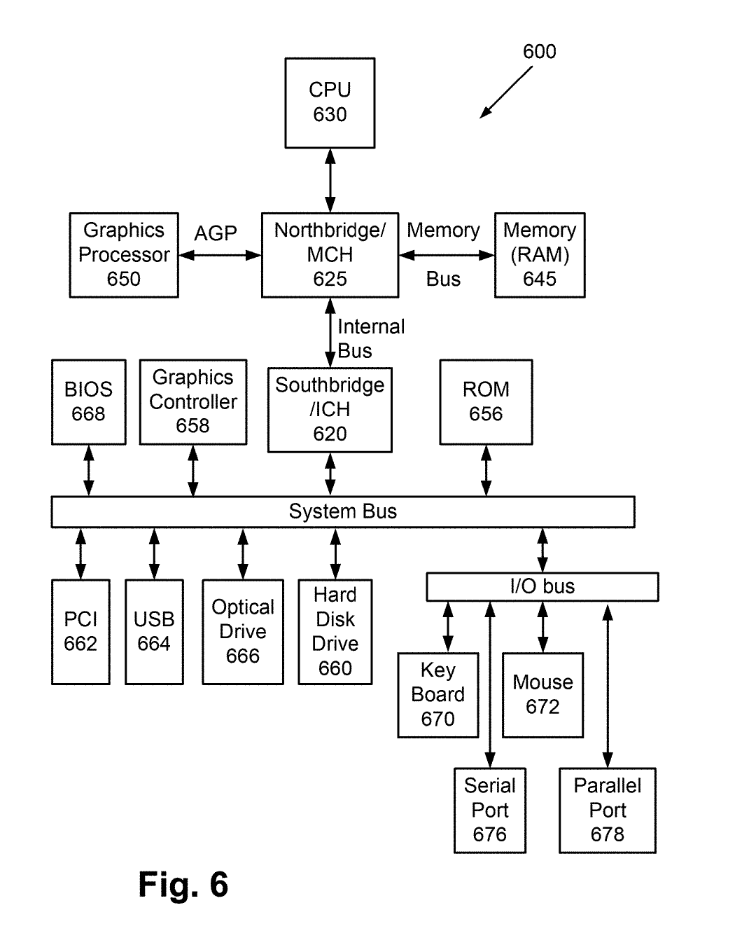

The exemplary circuit elements described in the context of the present disclosure can be replaced with other elements and structured differently than the examples provided herein. Moreover, circuitry configured to perform features described herein can be implemented in multiple circuit units (e.g., chips), or the features can be combined in circuitry on a single chipset, as shown in FIG. 6. The chipset of FIG. 6 can be implemented in conjunction with either electronic device 400 or computing device 500 described above with reference to FIGS. 4 and 5, respectively.

FIG. 6 shows a schematic diagram of a data processing system, according to aspects of the disclosure described herein for performing menu navigation, as described above. The data processing system is an example of a computer in which code or instructions implementing the processes of the illustrative embodiments can be located.

In FIG. 6, data processing system 600 employs an application architecture including a North Bridge and Memory Controller Hub (NB/MCH) 625 and a South Bridge and Input/Output (I/O) Controller Hub (SB/ICH) 620. The CPU 630 is connected to NB/MCH 625. The NB/MCH 625 also connects to the memory 645 via a memory bus, and connects to the graphics processor 650 via an Accelerated Graphics Port (AGP). The NB/MCH 625 also connects to the SB/ICH 620 via an internal bus (e.g., a unified media interface or a direct media interface). The CPU 630 can contain one or more processors and even can be implemented using one or more heterogeneous processor systems.

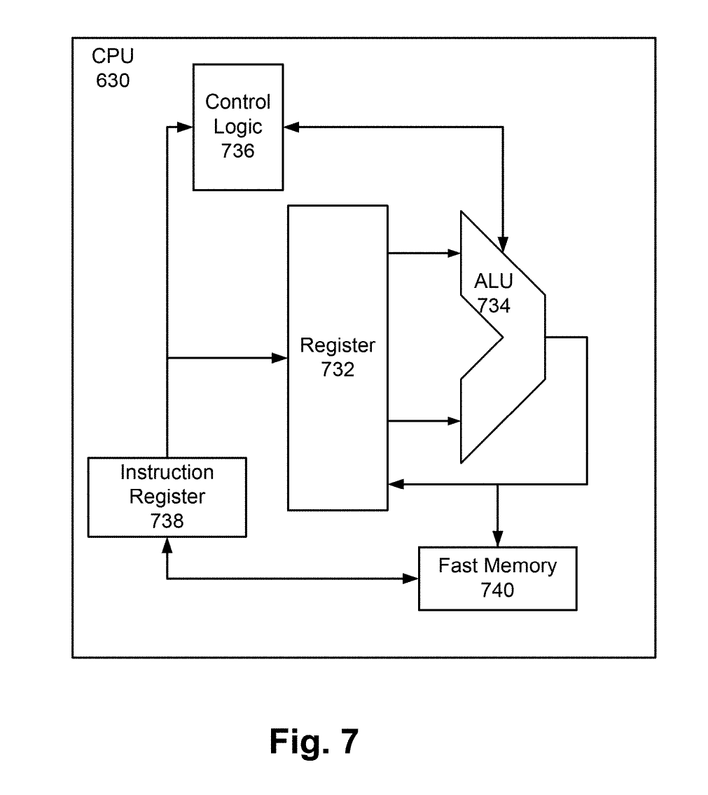

For example, FIG. 7 shows one implementation of CPU 630. In one implementation, an instruction register 738 retrieves instructions from a fast memory 740. At least part of these instructions are fetched from an instruction register 738 by a control logic 736 and interpreted according to the instruction set architecture of the CPU 630. Part of the instructions can also be directed to a register 732. In one implementation the instructions are decoded according to a hardwired method, and in another implementation the instructions are decoded according to a microprogram that translates instructions into sets of CPU configuration signals that are applied sequentially over multiple clock pulses. After fetching and decoding the instructions, the instructions are executed using an Arithmetic Logic Unit (ALU) 734 that loads values from the register 732 and performs logical and mathematical operations on the loaded values according to the instructions. The results from these operations can be fed back into the register 732 and/or stored in a fast memory 740. According to aspects of the disclosure, the instruction set architecture of the CPU 630 can use a Reduced Instruction Set Computer (RISC), a Complex Instruction Set Computer (CISC), a vector processor architecture, or a Very Long Instruction Word (VLIW) architecture. Furthermore, the CPU 630 can be based on the Von Neuman model or the Harvard model. The CPU 630 can be a digital signal processor, an FPGA, an ASIC, a PLA, a PLD, or a CPLD. Further, the CPU 630 can be an x86 processor by Intel or by AMD; an ARM processor; a Power architecture processor by, e.g., IBM; a SPARC architecture processor by Sun Microsystems or by Oracle; or other known CPU architectures.

Referring again to FIG. 6, the data processing system 600 can include the SB/ICH 620 being coupled through a system bus to an I/O Bus, a ROM 656, Universal Serial Bus (USB) port 664, a flash Binary Input/Output System (BIOS) 668, and a graphics controller 658. PCI/PCIe devices can also be coupled to SB/ICH 620 through a PCI bus 662.

The PCI devices can include, for example, Ethernet adapters, add-in cards, and PC cards for notebook computers. The HDD 660 and CD-ROM 666 can use, for example, an Integrated Drive Electronics (IDE) or Serial Advanced Technology Attachment (SATA) interface. In one implementation the I/O bus can include a Super I/O (SIO) device.

Further, the HDD 660 and optical drive 666 can also be coupled to the SB/ICH 620 through a system bus. In one implementation, a keyboard 670, a mouse 672, a parallel port 678, and a serial port 676 can be connected to the system bus through the I/O bus. Other peripherals and devices can be connected to the SB/ICH 620 using a mass storage controller such as SATA or PATA, an Ethernet port, an ISA bus, a LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry, or based on the requirements of the intended back-up load to be powered.

The functions and features described herein can also be executed by various distributed components of a system. For example, one or more processors can execute these system functions, wherein the processors are distributed across multiple components communicating in a network. The distributed components can include one or more client and server machines, which can share processing, such as a cloud computing system, in addition to various human interface and communication devices (e.g., display monitors, smart phones, tablets, Personal Digital Assistants (PDAs)). The network can be a private network, such as a LAN or WAN, or can be a public network, such as the Internet. Input to the system can be received via direct user input and received remotely either in real-time or as a batch process. Additionally, some implementations can be performed on modules or hardware not identical to those described. Accordingly, other implementations are within the scope that can be claimed.

The functions and features described herein may also be executed by various distributed components of a system. For example, one or more processors may execute these system functions, wherein the processors are distributed across multiple components communicating in a network. For example, distributed performance of the processing functions can be realized using grid computing or cloud computing. Many modalities of remote and distributed computing can be referred to under the umbrella of cloud computing, including: software as a service, platform as a service, data as a service, and infrastructure as a service. Cloud computing generally refers to processing performed at centralized locations and accessible to multiple users who interact with the centralized processing locations through individual terminals.

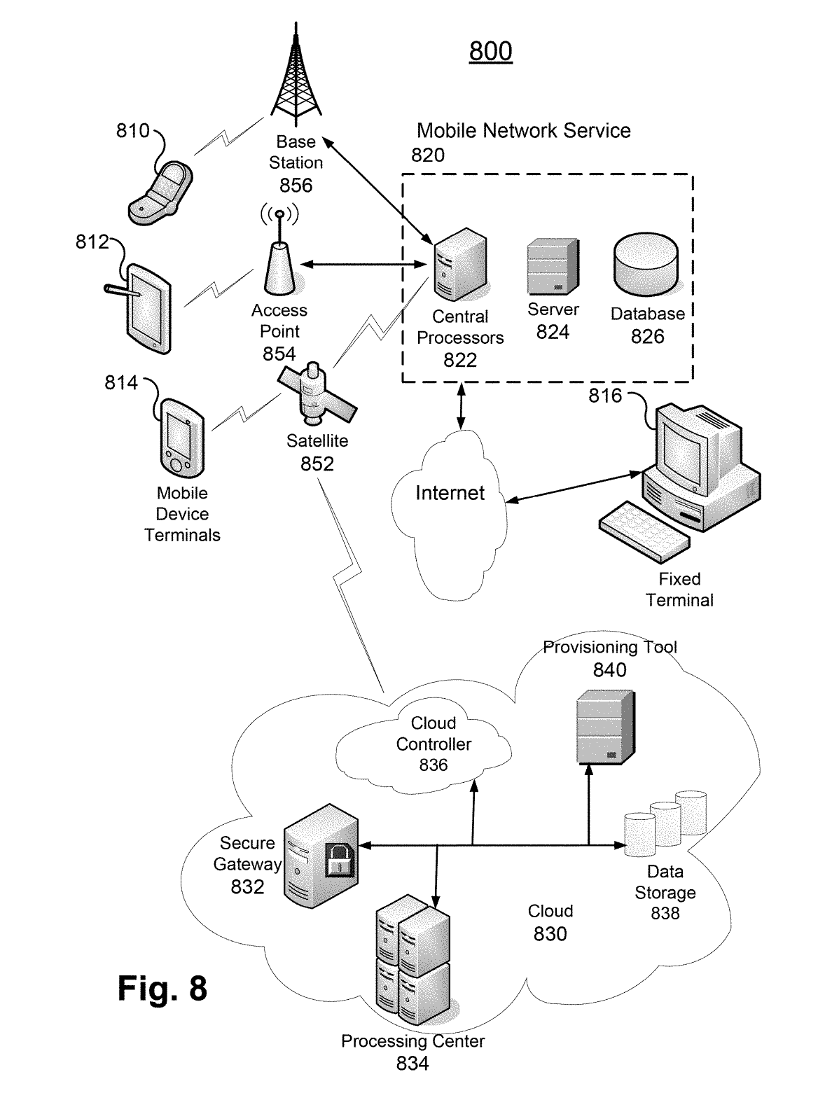

FIG. 8 illustrates an example of a cloud computing system 800, wherein users access the cloud through mobile device terminals or fixed terminals that are connected to the Internet. One or more of the devices illustrated as servers 160, 160A, and 160B, and mobile device 312, as well as platform 130 and applications 110 could be used in the cloud computing system 800 illustrated in FIG. 8.

The mobile device terminals can include a cell phone 810, a tablet computer 812, and a smartphone 814, for example. The mobile device terminals can connect to a mobile network service 820 through a wireless channel such as a base station 856 (e.g., an Edge, 3G, 4G, or LTE Network), an access point 854 (e.g., a femto cell or WiFi network), or a satellite connection 852. In one implementation, signals from the wireless interface to the mobile device terminals (e.g., the base station 856, the access point 854, and the satellite connection 852) are transmitted to a mobile network service 820, such as an EnodeB and radio network controller, UMTS, or HSDPA/HSUPA. Mobile users' requests and information are transmitted to central processors 822 that are connected to servers 824 to provide mobile network services, for example. Further, mobile network operators can provide service to mobile users for authentication, authorization, and accounting based on home agent and subscribers' data stored in databases 826, for example. The subscribers' requests are subsequently delivered to a cloud 830, such as cloud 120, 220, and 331 through the Internet.

A user can also access the cloud 830 through a fixed terminal 816, such as a desktop or laptop computer or workstation that is connected to the Internet via a wired network connection or a wireless network connection. The mobile network service 820 can be a public or a private network such as an LAN or WAN network. The mobile network service 820 can be wireless such as a cellular network including EDGE, 3G and 4G wireless cellular systems. The wireless mobile network service 820 can also be Wi-Fi, Bluetooth, or any other wireless form of communication that is known.

The user's terminal, such as a mobile user terminal and a fixed user terminal, provides a mechanism to connect via the Internet to the cloud 830 and to receive output from the cloud 830, which is communicated and displayed at the user's terminal. In the cloud 830, a cloud controller 836 processes the request to provide users with the corresponding cloud services. These services are provided using the concepts of utility computing, virtualization, and service-oriented architecture.

In one implementation, the cloud 830 is accessed via a user interface such as a secure gateway 832. The secure gateway 832 can for example, provide security policy enforcement points placed between cloud service consumers and cloud service providers to interject enterprise security policies as the cloud-based resources are accessed. Further, the secure gateway 832 can consolidate multiple types of security policy enforcement, including for example, authentication, single sign-on, authorization, security token mapping, encryption, tokenization, logging, alerting, and API control. The cloud 830 can provide to users, computational resources using a system of virtualization, wherein processing and memory requirements can be dynamically allocated and dispersed among a combination of processors and memories to create a virtual machine that is more efficient at utilizing available resources. Virtualization creates an appearance of using a single seamless computer, even though multiple computational resources and memories can be utilized according to increases or decreases in demand. In one implementation, virtualization is achieved using a provisioning tool 840 that prepares and equips the cloud resources, such as the processing center 834 and data storage 838 to provide services to the users of the cloud 830. The processing center 834 can be a computer cluster, a data center, a main frame computer, or a server farm. In one implementation, the processing center 834 and data storage 838 are collocated.

Embodiments described herein can be implemented in conjunction with one or more of the devices described above with reference to FIGS. 4-8. Embodiments are a combination of hardware and software, and processing circuitry by which the software is implemented.

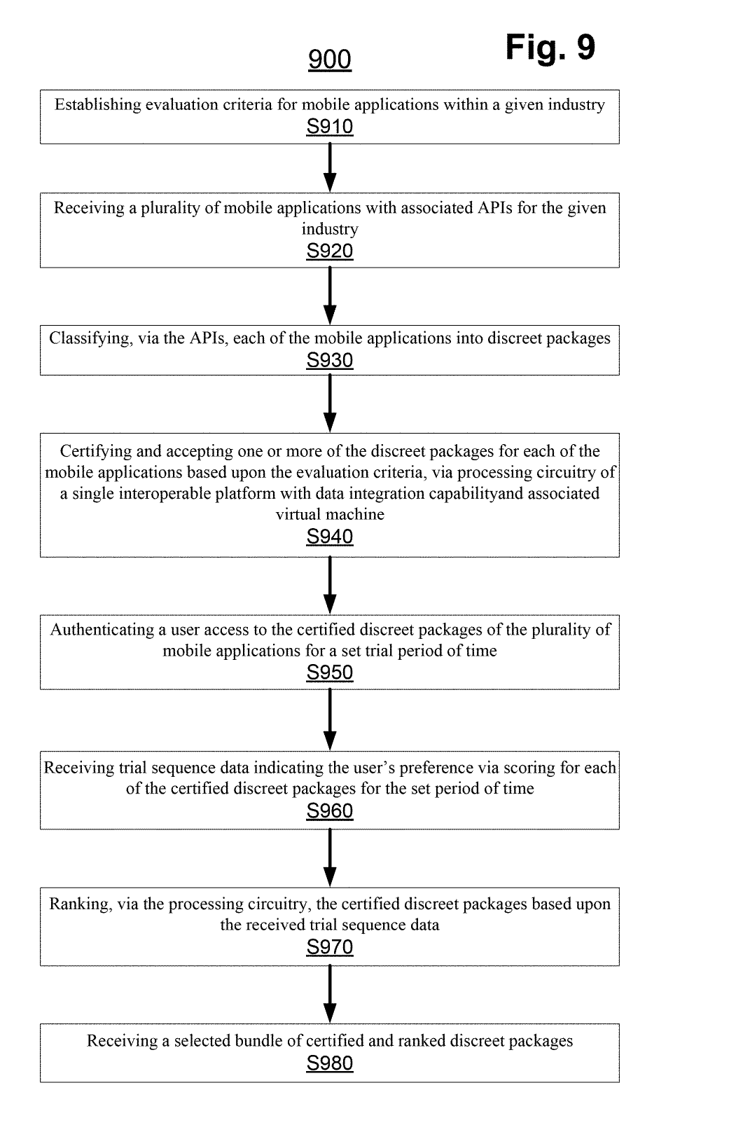

FIG. 9 illustrates an exemplary algorithmic flowchart for a method 900 of comparing mobile applications according to an aspect of the present disclosure. Method 900 includes programmable computer-executable instructions, that when used in combination with the above-described systems 200 and 300 and the above-described hardware devices, carry out the steps of method 900. The hardware description above, exemplified by any one of the structural examples illustrated in FIGS. 4-6 constitutes or includes specialized corresponding structure that is programmed or configured to perform the algorithm illustrated in FIG. 9. For example, the algorithm illustrated in FIG. 9 can be completely performed by the single device illustrated in FIG. 4 or 5, or the chipset illustrated in FIG. 6. The algorithm can also be completely performed in a shared manner distributed over the circuitry of any plurality of devices in a cloud computing system, such as cloud computing system 800.

FIG. 9 is a flowchart for a method 900 of comparing mobile applications, using the architectural framework 300 and one or more of the computing systems 500, 600, or 800 described above. In step S910, evaluation criteria for mobile applications is established within a given industry, such as the medical and health industry. Evaluation criteria can be determined from an applicable standards body and/or from professionals within the given industry.

In step S920, a plurality of mobile applications with their associated APIs is received for the given industry. The APIs provide the tools by which each mobile application can be integrated with other mobile applications.

In step S930, each of the mobile applications is classified into discreet packages, via each associated API. A discreet package is a discreet feature of a mobile application which can function as a separate entity, apart from other discreet packages within the same mobile application.

In step S940, one or more of the discreet packages for each of the mobile applications is certified and accepted, via processing circuitry of a single interoperable platform with data integration capability and associated virtual machine. The certifying and accepting is based upon the evaluation criteria. In an embodiment, the single interoperable platform has electronic data integration capability.

In step S950, a user is authenticated for access to the certified discreet packages of the plurality of mobile applications for a set trial period of time. In an embodiment, a user is given an equal amount of time to try out at least two mobile applications or to try out at least two discreet packages of a mobile application.

In step S960, trial sequence data is received indicating the user's preference via scoring for each of the mobile applications or the discreet packages for the set period of time. In an embodiment, the user is given additional mobile applications or additional discreet packages to try out if an acceptable application or discreet package is not indicated by the user.

In step S970, the certified discreet packages are ranked, via the processing circuitry, based upon the received trial sequence data. In an embodiment, step S970 integrates the evaluation criteria from applicable professionals and/or a professional body with the user evaluations.

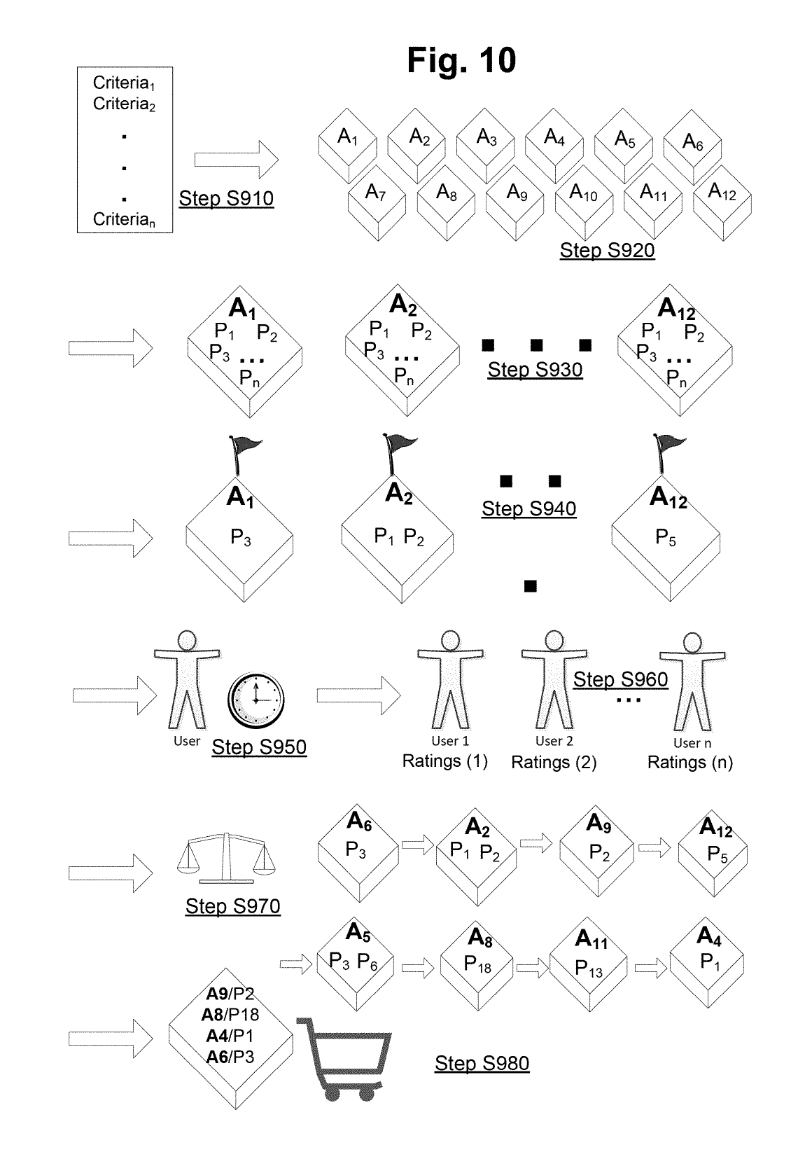

In step S980, a selected bundle of certified and ranked discreet packages is received. In an embodiment, a user may have selected one or more discreet packages from a plurality of mobile applications. For example in FIG. 10, a user has selected a discreet package₂ from application₉, a discreet package₁₈ from applications, a discreet packag₁ from application₄, and a discreet package₃ from application₆ as a customized mobile application for purchase. A pricing schedule can be established for each discreet package for each mobile application. In an embodiment, a volume discount is given for a predetermined number of purchased discreet packages.

FIG. 10 is a pictorial illustration of method 900 for comparing mobile applications, in which evaluation criteria is established for a particular industry (step S910), a plurality of mobile applications and their associated APIs are received (step S920), each mobile application is classified into discreet packages, via their respective APIs (step S930), and one or more of the discreet packages are certified and accepted onto a single interoperable platform, based upon the evaluation criteria and the user ratings (step S940).

The acceptance onto the operating platform can include consideration of a third-party validation of the mobile applications, such as an official governing body rating or a recommendation by an industry-related practitioner or organization. After acceptance, the accepted mobile application can be output to an interface connected to a server for subsequent transmission. Each of the mobile applications shares an API with the single interoperable platform.

In step S950, an authenticated user is granted access to multiple industry-related mobile applications and their associated discreet packages. The user has a set period of time to try out each of the discreet packages of the mobile applications and determine if he/she finds the discreet packages or applications useful, helpful, interesting, etc. The same set period of time is allotted for each mobile application or discreet package.

Trial sequence data is received in step S960, which indicates the user's preference for each of the certified discreet packages of the mobile applications. The user can compare and rate each of the discreet packages or mobile applications using a set of industry-related criteria. Criteria for the user to evaluate might include clear instructions, user-friendly input, organized format, length of time to complete, reliability of connected products (e.g. blood pressure kit), and real-time results pushed to a third party (e.g. doctor's office).

The received information related to the user can be used to output a recommendation for one or more of the mobile applications. When a mobile application is received, it is determined if any data fields are currently unpopulated, and obtain data from a record server for the unpopulated data fields. Information related to the user can also be received and output to data fields in another mobile application. The received data from some of the mobile applications can be used by the user to integrate the data into a single application.

A ranking of the certified discreet packages is generated based on the established evaluation criteria and the received trial sequence data in step S970. The ranking can be generated using one of a pairwise ranking, a list-wise correlation, or a multiple alignment technique. In step S980, one or more selected discreet packages are received in an order from a user.

Method 900 can also include de-identifying protected information from the received trial sequence data. The received data from some of the mobile applications can be clustered, from which new related mobile applications can be recommended to the user. In an embodiment, the industry-related mobile applications pertain to the medical and health industry.

A mobile application network system includes an interoperable platform and associated virtual machine language, an operating system connected to the interoperable platform, and a server, wherein the mobile application network system includes circuitry configured to execute the steps described above.

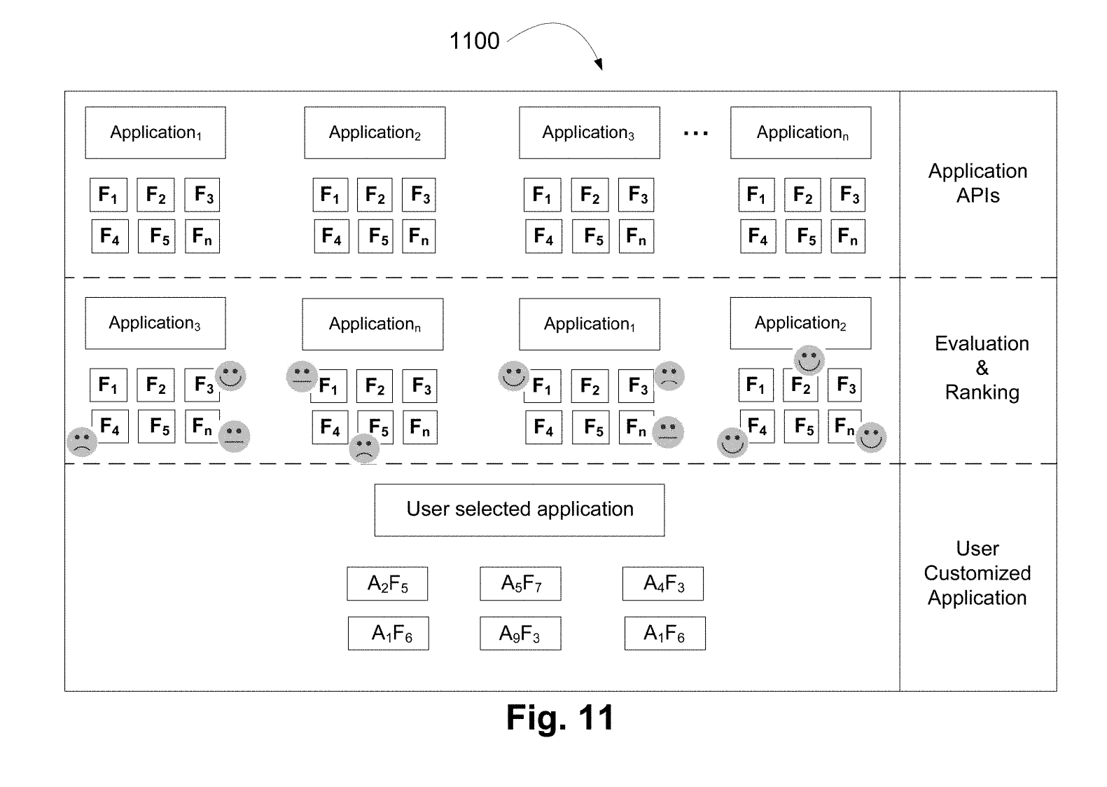

FIG. 11 is an illustration of a user interface 1100 provided to a user device. FIG. 11 is illustrated as a single screen. However, multiple screens can be utilized for the various stages of presentation, evaluation, and selection.

The mobile application mApp for a particular industry, such as the health and medical industry is provided. The first row of the user interface 1100 illustrates Application₁ through Applicationₙ that are available to a user and their associated functions, such as the discreet packages associated with each application illustrated in FIG. 10.

The second row of the user interface 1100 illustrates results of evaluating and ranking the multiple applications. Each of the functions of each application is evaluated. For example, the functionality F1 of Application1 is evaluated by a relevant association according to pre-established industrial criteria. Each function of each application is also evaluated by one or more users according to usability criteria. The evaluations are combined with other associated factors for a total evaluation score of each application. The applications are subsequently ranked according to their total evaluation score, such as the ranking illustrated in the second row of FIG. 11.

The ranking illustrated in the second row of FIG. 11 is constantly refreshed based upon new and revised rankings provided by association ratings and user ratings. Links can also be provided for each rating with comments and/or emoji from the association or user to substantiate the associated ratings.

The third row of FIG. 11 illustrates a user customized application that was selected from one or more applications and associated functionalities that were available. The user customized application is a bundle of selected functions that a user deemed to be useful. Only the selected functions are purchased by the user, rather than requiring the user to purchase an entire application for a particular desirable function. In an embodiment, the user interface can be linked to a user profile, such that one or more application functions can be suggested to the user. In an example given for illustrative purposes only, a user can customize a mobile health application based upon a particular condition, disease, and/or disorder.

Embodiments described herein for a mobile interface platform offer several advantages. A mobile application and its associated functions can be authenticated by individuals and/or associations within the particular industry. A safe and risk-free mobile application market is provided to users, which is also available for professionals to recommend to their customers/clients/patients. Certifications of the application functions are based on professional industrial criteria and usability criteria. The dual industry rating and user rating result in a fair rating methodology. Ranking of the application functions increases competitiveness, which is realized in the real-time rankings presented in the user interface 1100. A positive user experience is provided by offering a lower cost from purchasing separate functions of an application instead of purchasing the entire application, freedom to pick and choose, and an available volume discount.

Embodiments are given herein, in which the methods and systems described are utilized within the medical and health industry. However, the embodiments discussed (or other analogous embodiments) can also be applied to most other industries, including but not limited to the auto, music, entertainment, food, exercise, education, and travel industries without departing from the scope of the present disclosure.

In a first embodiment, informational data related to a patient's medical condition is received from a server, such as the server 160A illustrated in FIG. 3B. Based upon the medical condition of the patient derived from the received informational data, a recommendation is forwarded to the patient for one or more mobile health applications or one or more discreet packages of a mobile health application(s) from the medical and health platform, such as platform 130. One or more services of relevant medical applications may also be forwarded to the patient.

In a second embodiment, informational data related to a patient's medical condition is received from a server. The received informational data is outputted into corresponding data fields of a mobile health application.

In a third embodiment, a mobile health application is received by a host device. The host device determines data fields that are currently unpopulated in a mobile health application. The host device obtains data that corresponds to the unpopulated fields from a server that has an applicable record, such as an electronic record server. For example, a mobile health allergy application for a patient may contain data that a mobile health prescription application needs for that patient. The relevant data from the allergy application is obtained from the server by the host device, and the data is populated into the prescription application.

In a fourth embodiment, an authentication to access a plurality of mobile health applications containing de-identified data sets is received for a predetermined trial period. Following the trial period, pairwise trial sequence data indicating a user's preference for each of the mobile health applications with respect to the other applications is received. A ranking for each of the applications is generated, based on the pairwise trial sequence data.

In a fifth embodiment, a marketplace service functions as a pEHR. The pEHR gathers new medical data collected with each new application download. If a user downloads a new application that requires information from the user that had not been requested previously, such as blood type, the pEHR expands its data collection for that user by one new field, i.e. blood type. The pEHR makes it available to subsequent applications when requested, if approved by the user. This bi-directional nature prevents the need to create an EHR that anticipates all information requests before the need arises.

In a sixth embodiment, the interoperable platform is an open platform. Subscribers, developers, and users have access to the platform for potential future mobile applications. For example, basic demographic information about prospective users can be uploaded. Also, basic information about mobile application services and features can be shared.

In addition to integrating with other health applications and systems, a mobile medical or health application should have certain features in order to be useful, helpful, and effective. The application needs to be fit for the purpose, whether it is intended for a patient, physician, healthcare provider, or clinician. It should also be attractive and adapted to the environment of the user. The same general purpose application could have several different versions for different users, as well as different versions for the same type of user. For example, a mobile health application's purpose may be to manage health-related appointments. The patient would have use for such an application, as well as the individual health entities (e.g. doctor's offices, diagnostic or laboratory facilities, or physical therapy facilities). In addition, such an application geared to the patient could have different versions for a child, young adult, and senior adult. Other combinations of mobile health applications and versions of those applications are contemplated by embodiments described herein.

The mobile health application should store the data securely and according to any local laws and regulations. For example, the Health Insurance Portability and Accountability Act (HIPAA) has five regulations with standards or rules for privacy, security, transactions and code sets, unique identifiers, and enforcement of the Health Information Technology for Economic and Clinical Health (HITECH) Act. The first HIPAA regulation is the Privacy Rule, which mandates the protection and privacy of all health information. The Privacy Rule defines the authorized uses and disclosures of “individually-identifiable” health information. The Privacy Rule sets requirements for how Protected Health Information (PHI), in any form or medium is controlled. The second HIPAA regulation is the Security Rule, which mandates the security of EMRs. The Security Rule addresses the technical aspects of protecting electronic health information, including administrative security, physical security, and technical security. The third HIPAA regulation is the Transactions and Code Set (TCS) Rule, which addresses the use of predefined transaction standards and code sets for communications and transactions in the health-care industry. The fourth HIPAA regulation is the Unique Identifiers Rule, which has three unique identifiers used for covered entities in HIPAA transactions to promote standardization, efficiency, and consistency. The fifth HIPAA regulation is the Enforcement Rule, which stems from the HITECH Act. The Enforcement Rule expands the scope of the Privacy and Security Rules, and increases the reach and penalties for HIPAA violations.

Protected Health Information (PHI) is any information about the health status, a provision of health care, or a payment for health care that can be linked to a specific individual. This is interpreted to include any part of a patient's medical record or payment history. PHI is often sought out in datasets for de-identification before researchers share the dataset publicly. Removing PHI from a dataset preserves privacy for the research participants. Under HIPAA, PHI is based on 18 identifiers that must be treated with special care. Those 18 identifiers include names, all geographical identifiers smaller than a state, dates directly related to an individual, phone numbers, fax numbers, email addresses, US Social Security Administration (SSA) numbers or other personal identifying numbers, medical record numbers, health insurance beneficiary numbers, account numbers, certificate/license numbers, vehicle identifiers and serial numbers, device identifiers and serial numbers, web Uniform Resource Locators (URLs), Internet Protocol (IP) address numbers, biometric identifiers, full face photographic images, and any other unique identifying number, characteristic, or code.

De-identification under the HIPAA rule occurs when data has been stripped of the above common identifiers by either removing all 18 specific identifiers, or by obtaining the expertise of an experienced statistical expert to validate and document the statistical risk of re-identification as being very small, according to a statistical method. Anonymization is a process in which PHI elements are eliminated or manipulated with the purpose of hindering the possibility of going back to the original data set. This involves removing all identifying data to create unlinkable data. De-identified data is coded, with a link to the original, fully identified data set. Links exist in coded de-identified data, making the data considered indirectly identifiable and not anonymized. Coded de-identified data is not protected by the HIPAA Privacy Rule, but is protected under the Common Rule. When de-identification and anonymization are used together, health care data can be used in larger increments and still abide by HIPAA regulations.

A mobile health application should also have any necessary certifications in place. The realm of mobile health applications is very large, and any necessary certifications will also span a large spectrum. Therefore, a good mobile health application will have any required certifications readily visible.

A mobile health application becomes much more valid when it is supported by any relevant scientific backing. For example, a mobile health application may pertain to a dental process. Therefore, a valid scientific backing might include a statement that the American Dental Association has approved or certified a particular version number and date of the application. Many other purposes and associated backings are contemplated by embodiments described herein.

A mobile health application should be easily implemented. Many applications, including applications other than mobile or health-related applications may have a valuable and/or interesting purpose, but are difficult to implement. As a result, the user gives up and is disappointed that it did not fulfill his/her need.

A mobile application needs to be credible in order for professionals within that industry to prescribe, recommend, or endorse the application. If all HIPAA regulations were adhered to in an application, it would likely render the application credible. For the medical and health industry, a physician is likely to prescribe a particular mobile application if the application has a credible health-related backing, such as the US FDA. However, not all applications originate from the United States, and those application authors may not bother to seek FDA approval or adhere to HIPAA regulations. In addition, the waiting period for FDA approval can be lengthy. An embodiment of the invention includes a list of criteria, established by the governing platform, which all mobile applications must meet. Another embodiment includes not publishing or going live with the application until it has met those criteria and is approved by the platform.

As time progresses with the approval of mobile applications by a platform, a natural clustering of applications will occur. In the health and medical industry, certain medical conditions may likely catalyze a cluster of applications around that condition. For example, a diabetic cluster may form, which might include a glucose monitoring application, a diet application, a social network application, and a medical supplies ordering application. A second example includes an electronic health and nutrition exchange. A third example includes an auto clustering, in which applications may cluster in the areas of race cars, antique cars, trucks, and green cars to name just a few. A fourth example includes a music cluster, in which applications may cluster by music genre. To facilitate any type of clustering, the platform marketplace could provide cluster-specific suggestions that match profiles of similar users.

An extension of clustering is also provided by embodiments of the present disclosure. Mobile applications are integrated together by industry onto a platform. Clustering occurs and cluster-specific suggestions can be made, as discussed above. A user's data can also be applied against multiple applications. As a result, different applications can be cross-matched to a user. In addition, overlapping of more than one cluster can occur. For example, in the health and medical industry, a patient/user may have more than one medical condition, especially with chronic symptoms. Therefore, this particular user could have several application links, from user-designated applications and from marketplace-suggested applications. In addition, marketplace arrangements can be set up for application bundle purchases.

Embodiments of the present disclosure provide an evaluation and validation of mobile applications that focus on increasing the number of high quality and safe mobile applications, and promote collaboration between practitioners, innovators, developers, and academics. Embodiments described herein also create a community of interest to drive ideas into practice.

With regard to the medical and healthcare industry, the rise of the hyper-connectivity allows real-time patient monitoring through wearable mobile applications that are connected wirelessly to machines at a physician's office through e-health applications. This allows real-time interactions between patients and their healthcare providers to improve health outcomes and ultimately save lives.

The potential impact of mobile health application prescribing for a patient's care has the following advantages. For patients, embodiments described herein improve access to health care, improve quality of health care, decrease hospitalization, and decrease costs. This allows a patient to keep abreast of changes in his/her health. For physicians, embodiments described herein provide personalized treatment plans, improve patient satisfaction and outcomes, and increase referrals. For health care providers, embodiments described herein move towards a more patient-centric or performance-based model of care delivery, and promote the mobile applications that make patients engage in their own health management between appointments.

The mHi platform allows physicians to make recommendations from a trustworthy portal mobile health application. In an example, “prescribable mobile apps” are evaluated and approved, via the mHi platform. Prescribable apps assist physicians in prescribing one or more prescriptions to patients to provide continuity of their health care in between physician visits, especially to patients having one or more chronic diseases.

Portal access to mHi platform allows selection of one or more prescribable apps for patients during the period of in-between physician visits or hospitalizations. In addition, prescribable apps could provide information of potential adverse drug interactions.

Embodiments described herein provide several benefits and advantages. The single interoperable platform provides mobile applications that have met established evaluation criteria and have been rated by its users. This provides a level of confidence for users, professionals, and organizations within a particular industry. In the medical health industry, a level of confidence is established to prescribe a certified application that has been marked as a trustworthy application by a balanced, neutral, and fair platform. Physicians have a level of confidence to prescribe related applications along with a medical regimen.

In the medical industry, a certified prescription mobile application can be recommended by a physician along with a pharmaceutical prescription to a patient. This can guide patients in between physician visits, fully inform patients of associated instructions such as conditions for taking the medication, and any symptoms or warning signs that should be reported to the attending physician.

Embodiments described herein provide a mechanism for purchasing only those features of interest. Users can purchase one or more discreet packages from any number of mobile applications, instead of purchasing the entire mobile applications. This provides customization at an affordable price.

Embodiments described herein include the following aspects.

(1) A method of evaluating mobile applications includes establishing evaluation criteria for mobile applications within a given industry; receiving a plurality of mobile applications with associated APIs for the given industry; classifying, via the APIs, each of the mobile applications into discreet packages; certifying and accepting one or more of the discreet packages for each of the mobile applications based upon the evaluation criteria, via processing circuitry of a single interoperable platform with data integration capability and associated virtual machine; authenticating a user access to the certified discreet packages of the plurality of mobile applications for a set trial period of time; receiving trial sequence data indicating the user's preference via scoring for each of the certified discreet packages for the set period of time; ranking, via the processing circuitry, the certified discreet packages based upon the received trial sequence data; and receiving a selected bundle of certified and ranked discreet packages.

(2) The method of (1), wherein the mobile applications are from the health and medical industry.

(3) The method of either (1) or (2), further includes receiving a comparison from the user of a plurality of certified discreet packages for the set trial period of time.

(4) The method of any one of (1) through (3), further includes receiving a rating from the user of the compared certified discreet packages via a set of industry-related criteria.

(5) The method of any one of (1) through (4), wherein the ranking is generated via one of a pairwise ranking, a list-wise correlation, or a multiple alignment technique.

(6) The method of any one of (1) through (5), wherein the certifying and accepting includes consideration of a third-party validation of the discreet packages.

(7) The method of any one of (1) through (6), further includes outputting the certified and accepted discreet packages to an interface connected to a server from which the discreet packages may be transmitted.

(8) The method of any one of (1) through (7), further includes receiving information related to the user and outputting a recommendation for one or more discreet packages, based upon the received information.

(9) The method of any one of (1) through (8), further includes receiving information related to the user and outputting some of the received information that corresponds to data fields in another mobile application.

(10) The method of any one of (1) through (9), further includes determining, when a mobile application is received, data fields that are currently unpopulated in the received mobile application; and obtaining data corresponding to the unpopulated data fields from a record server.

(11) The method of any one of (1) through (10), wherein each of the mobile applications share their associated API with the single operating platform.

(12) The method of any one of (1) through (11), further includes de-identifying protected information from the received trial sequence data.

(13) The method of any one of (1) through (12), further includes receiving data from some of the mobile applications used by the user, and integrating the data into a single application.

(14) The method of any one of (1) through (13), further includes clustering the received data from some of the mobile applications; and recommending one or more new related mobile applications to the user from the clustered received data.

(15) The method of any one of (1) through (14), further includes receiving a customized mobile application having a plurality of certified discreet packages, which is based upon selected criteria from the user.

(16) The method of any one of (1) through (15), further includes receiving a customized mobile application having a plurality of certified discreet packages, which is based upon selected criteria from a service provider.

(17) The method of any one of (1) through (16), further includes receiving a customized mobile application having a plurality of certified discreet packages, which is based upon a prescribable regimen for one or more users.

(18) A mobile application network includes an interoperable platform and associated virtual machine; an operating system connected to the interoperable platform; a server connected to the operating system; and processing circuitry configured to establish evaluation criteria for mobile applications within a given industry, receive a plurality of mobile applications with associated APIs within the given industry, classify, via the APIs, each of the mobile applications into discreet packages, certify and accept one or more of the discreet packages for each of the mobile applications based upon the evaluation criteria, via the interoperable platform with data integration capability and associated virtual machine, authenticate a user access to the certified discreet packages of the plurality of mobile applications for a set trial period of time, receive trial sequence data indicating the user's preference via scoring for each of the certified discreet packages for the set period of time, rank the certified discreet packages based upon the received trial sequence data, and receive a selected bundle of certified and ranked discreet packages.

(19) The mobile application network of (18), wherein the received trial sequence data includes an equal-time comparison by the user of at least two of the discreet packages according to industry-related criteria.

(20) The mobile application network of either (18) or (19), wherein the discreet packages verified and accepted by the interoperable platform include a third-party validation.

The foregoing discussion describes merely exemplary embodiments of the present disclosure. As will be understood by those skilled in the art, the present disclosure may be embodied in other specific forms without departing from the spirit or essential characteristics thereof. Accordingly, the disclosure is intended to be illustrative, but not limiting of the scope of the disclosure, as well as the claims. The disclosure, including any readily discernible variants of the teachings herein, defines in part, the scope of the foregoing claim terminology such that no inventive subject matter is dedicated to the public.

1. A method of evaluating mobile applications, the method comprising:
establishing evaluation criteria for mobile applications within a given industry;
receiving a plurality of mobile applications with associated Application Programming Interfaces (APIs) for the given industry;
classifying, via the APIs, each of the mobile applications into discreet packages;
certifying and accepting one or more of the discreet packages for each of the mobile applications based upon the evaluation criteria, via processing circuitry of a single interoperable platform with data integration capability and associated virtual machine;
authenticating a user access to the certified discreet packages of the plurality of mobile applications for a set trial period of time;
receiving trial sequence data indicating the user's preference via scoring for each of the certified discreet packages for the set period of time;
ranking, via the processing circuitry, the certified discreet packages based upon the received trial sequence data; and
receiving a selected bundle of certified and ranked discreet packages.

2. The method of claim 1, wherein the mobile applications are from the health and medical industry.

3. The method of claim 1, further comprising:
receiving a comparison from the user of a plurality of certified discreet packages for the set trial period of time.

4. The method of claim 3, further comprising:
receiving a rating from the user of the compared certified discreet packages via a set of industry-related criteria.

5. The method of claim 1, wherein the ranking is generated via one of a pairwise ranking, a list-wise correlation, or a multiple alignment technique.

6. The method of claim 1, wherein the certifying and accepting includes consideration of a third-party validation of the discreet packages.

7. The method of claim 6, further comprising:
outputting the certified and accepted discreet packages to an interface connected to a server from which the discreet packages may be transmitted.

8. The method of claim 1, further comprising:
receiving information related to the user and outputting a recommendation for one or more discreet packages, based upon the received information.

9. The method of claim 1, further comprising:
receiving information related to the user and outputting some of the received information that corresponds to data fields in another mobile application.

10. The method of claim 1, further comprising:
determining, when a mobile application is received, data fields that are currently unpopulated in the received mobile application; and
obtaining data corresponding to the unpopulated data fields from a record server.

11. The method of claim 1, wherein each of the mobile applications share their associated API with the single operating platform.

12. The method of claim 1, further comprising:
de-identifying protected information from the received trial sequence data.

13. The method of claim 1, further comprising:
receiving data from some of the mobile applications used by the user, and integrating the data into a single application.

14. The method of claim 13, further comprising:
clustering the received data from some of the mobile applications; and
recommending one or more new related mobile applications to the user from the clustered received data.

15. The method of claim 1, further comprising:
receiving a customized mobile application having a plurality of certified discreet packages, which is based upon selected criteria from the user.

16. The method of claim 1, further comprising:
receiving a customized mobile application having a plurality of certified discreet packages, which is based upon selected criteria from a service provider.

17. The method of claim 1, further comprising:
receiving a customized mobile application having a plurality of certified discreet packages, which is based upon a prescribable regimen for one or more users.

18. A mobile application network, comprising:
an interoperable platform with data integration capability and associated virtual machine;
an operating system connected to the interoperable platform;
a server connected to the operating system; and
processing circuitry configured to
establish evaluation criteria for mobile applications within a given industry,
receive a plurality of mobile applications with associated application programming interfaces (APls) within the given industry,
classify, via the APIs, each of the mobile applications into discreet packages,
certify and accept one or more of the discreet packages for each of the mobile applications based upon the evaluation criteria, via the interoperable platform and associated virtual machine,
authenticate a user access to the certified discreet packages of the plurality of mobile applications for a set trial period of time,
receive trial sequence data indicating the user's preference via scoring for each of the certified discreet packages for the set period of time,
rank the certified discreet packages based upon the received trial sequence data, and
receive a selected bundle of certified and ranked discreet packages.

19. The mobile application network system of claim 18, wherein the received trial sequence data includes an equal-time comparison by the user of at least two of the discreet packages according to industry-related criteria.

20. The mobile application network system of claim 19, wherein the discreet packages verified and accepted by the interoperable platform include a third-party validation.
